(19) United States
(12) Patent Application Publication
(10) Pub. No.: US 2014/0296593 A1
(43) Pub. Date: Oct. 2, 2014

(54) PROCESS FOR THE FLUID CATALYTIC CRACKING OF OXYGENATED HYDROCARBON COMPOUNDS FROM BIOLOGICAL ORIGIN

(71) Applicant: SHELL OIL COMPANY, Houston, TX (US)

(72) Inventors: Wei ZHU, DongYing (CN); Yinsuo CAI, DongYing (CN); YiBin LIU, Qingdao (CN); YongShan TU, Qingdao (CN); ChaoHe YANG, Qingdao (CN); Yunying QI, Sugar Land, TX (US); Robert Alexander LUDOLPH, Sugar Land, TX (US); James Lloyd JENKINS, Houston, TX (US); Theodorus Johannes BROK, Amsterdam (NL); Colin John SCHAVERIEN, Amsterdam (NL); Binghui LI, Sugar Land, TX (US)

(73) Assignee: SHELL OIL COMPANY, Houston, TX (US)

(21) Appl. No.: 14/227,694

(22) Filed: Mar. 27, 2014

(30) Foreign Application Priority Data
Mar. 28, 2013 (CN) 201310104639.X

(51) Int. Cl. C07C 7/10; C07C 4/06

(52) U.S. Cl. CPC C07C 7/10; C07C 4/06; C07C 2529/89
USPC 585/310

(57) ABSTRACT

A process for the fluid catalytic cracking of oxygenated hydrocarbon compounds from biological origin. The process comprises contacting a feed comprising the oxygenated hydrocarbon compounds from biological origin and an amount of sulphur with a fluid cracking catalyst at a temperature of equal to or more than 400° C. to produce a products stream. The process further comprises separating fluid cracking catalyst from the products stream and separating a light fraction from the products stream; and removing hydrogen sulphide from the light fraction by means of an amine treating process. Activated carbon is used to treat at least part of an amine solution used in the amine treating process or to treat at least part of the feed to the amine treating process.

### PRIORITY CLAIM

The present non-provisional application claims priority from Chinese application no. 201310104639.X, filed Mar. 28, 2013, the disclosures of which are incorporated herein by reference.

### TECHNICAL FIELD

The present disclosure relates to a process for the fluid catalytic cracking of oxygenated hydrocarbon compounds from biological origin.

### BACKGROUND

Fluid catalytic cracking (FCC) is an important conversion process in present oil refineries. It can be used to convert high-boiling hydrocarbon fractions derived from perude oils into more valuable products such as gasoline components (naphtha), fuel oils and (olefinic) gases (ethene, propene, butene, LPG).

With the diminishing supply of crude petroleum oil, use of renewable energy sources is becoming increasingly important for the production of liquid fuels. These fuels from renewable energy sources are often referred to as biofuels. Such renewable energy sources may also be used as feeds to a fluid catalytic cracking process.

For example, Tian Hua et al. in their article titled “Alternative Processing Technology for Converting Vegetable Oils and Animal Fats to Clean Fuels and Light Olefins”, published in the Chinese Journal of Chemical Engineering, vol. 16 (3), pages 394-400 (2008) describe the fluid catalytic cracking of pure feeds of vegetable oils or animal fats and co-feeds with vacuum gas oil (VGO).

In chapter 7 of Dr. Tian Hua's dissertation titled “Studies on Catalytic Cracking of Fatty Acid Esters”, available from the college of Chemistry and Chemical Engineering, China University of Petroleum (EastChina) since April 2010, Dr. Tian Hua describes that one of the main operation problems experienced when co-processing a 22wt % bio-feed (a mix of animal and vegetable oil including used cooking oil) with a normal FCC vacuum Gas Oil (VGO) in a commercial Fluid Catalytic Cracking (FCC) unit was severe solvent foaming in the LPG sulphur removal column. It is mentioned that oxygen containing derivatives may have been absorbed and condensed in the so-called LPG sweetening column, resulting in severe foaming problems for the sulphur removal solvent (N-methyl-diethanolamine). In the dissertation it is suggested that with the adjustment of operating conditions to an increased riser residence time and an increased riser top temperature (i.e. an increased reaction severity) emulsion formation and solvent foaming could be reduced.

From a commercial perspective, however, the suggested increased reaction severity is disadvantageous. Increased temperatures and increased residence times will increase the operating costs of an FCC unit. In addition—on a commercial scale—flexibility in reaction severity may be desired to allow one to change the type of product made to fit market demand.

It would be an advancement in the art to provide a process that may reduce the above described solvent foaming but does not require adjustment of the operating conditions of the FCC reactor(s).

### SUMMARY

Applicant carried out test-runs to establish whether or not part or all of the feed for a commercial FCC unit could be replaced by material of biologic origin, more especially oils and fats of plant or animal origin.

During the test-runs it appeared that when changing the feed in a large (3000 barrels/day) integrated FCC unit from a completely petroleum-derived feed to a feed that comprises a petroleum-derived feed and a certain amount of biofeed (in this case more especially 10 wt % of used cooking oil or 10 wt % of tallow oil) immediately problems occurred in the amine treaters that are used to remove hydrogen sulphide from light product streams (such as for example dry gas and LPG). In the amine treaters highly undesired stable foams, sometimes in combination with emulsions, were formed. These foams/emulsions can be highly deleterious for the contact between the amine and the dry gas and/or LPG, which may result in insufficient removal of hydrogen sulphide. When the addition of biofeed was stopped, these problems disappeared.

It has now been advantageously found that the above described problems in an FCC process caused by the addition of biofeed to a petroleum-derived FCC feed may be overcome by passing the amine solution or a slipstream of the amine solution or amine treater feed or a slipstream of amine treater feed over an activated carbon filter.

In some embodiments, there is provided a process for the fluid catalytic cracking of oxygenated hydrocarbon compounds from biological origin. The process comprises contacting a feed comprising the oxygenated hydrocarbon compounds from biological origin and an amount of sulphur with a fluid cracking catalyst at a temperature of equal to or more than 400° C. to produce a products stream. The process further comprises separating fluid cracking catalyst from the products stream and separating a light fraction from the products stream; and removing hydrogen sulphide from the light fraction by means of an amine treating process, in which amine treating process activated carbon is used to treat at least part of an amine solution used in the amine treating process or to treat at least part of the feed to the amine treating process.

In some embodiments, the oxygenated hydrocarbon compounds from biological origin are derived from plant oil, animal fat or used cooking oil. In some embodiments, the amount of oxygenated hydrocarbon compounds is up till 65 vol % of the total feed, preferably between 1 and 45 vol %, more preferably between 2 and 35 vol %, even more preferably between 3 and 25 vol %.

In some embodiments, the light fraction is a fraction of the products stream comprising oxygen containing C1-C4 compounds having a biological origin having a boiling point equal to or less than 64° C. In some embodiments, the light fraction is a fraction comprising methanol, ethanol, propanol, butanol, formic acid, acetic acid, propionic acid, butanoic acid, acetone, formaldehyde, acetaldehyde and/or acrylaldehyde. In some embodiments, the light fraction is a C1-C2 fraction and/or a C3-C4 fraction. In some embodiments, the light fraction from the cracked products stream is obtained by feeding separated cracked products stream to a distillation column, fractionating the cracked products stream into an offgas fraction comprising C1-C4 compounds and at least one further fraction, optionally followed by separating fraction comprising the C1-C4 fraction into a fraction comprising mainly C1-C2 compounds and a fraction comprising mainly C3-C4 compounds.

In some embodiments, hydrogen sulphide is removed from a fraction comprising C1-C2 compounds and/or from a fraction comprising C3-C4 compounds. In some embodiments, steam is added to the feed/fluid cracking catalyst or steam is used to improve the separation of the catalyst from the products stream.

In some embodiments, the surface area of the activated carbon in the filter is more than 500 m²/g. In some embodiments, the activated carbon in the filter is physically or chemically activated. In some embodiments, a slipstream of the amine solution is treated, preferably a 5 to 50% slipstream of the amine solution, more preferably 10 to 25% slipstream. In some embodiments, the amine solution is a lean amine solution from the regenerator. In some embodiments, one or more antifoaming agents are used in the amine treating process. In some embodiments, a slipstream of 5 to 10 vol %/day of the amine solution is removed from the process and replaced with fresh amine solution. In some embodiments, the use of the activated carbon filter may prevent or reduce the formation of the stable foam and/or emulsions caused by the co-processing of biofuel.

Other advantages and features of embodiments of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following figures are included to illustrate certain aspects of the present disclosure, and should not be viewed as exclusive embodiments. The subject matter disclosed is capable of considerable modifications, alterations, combinations, and equivalents in form and function, as will occur to one having ordinary skill in the art and the benefit of this disclosure.

FIGS. 1a and 1b shows schematic diagrams of one embodiment of the process according to some aspects of the invention.

### DETAILED DESCRIPTION OF PREFERRED EMBODIMENTS

The present invention relates to a process for the fluid catalytic cracking of oxygenated hydrocarbon compounds from biological origin. Such fluid catalytic cracking (FCC) processes can suitably be carried out in fluid catalytic cracking (FCC) units comprising one or more fluid catalytic cracking (FCC) reactors.

Modern FCC units can operate continuous processes that may operate 24 hours a day for a period of two to four years. An extensive description of FCC technology can for example be found in “Fluid Catalytic Cracking technology and operations”, by Joseph W. Wilson, published by PennWell Publishing Company (1997) and “Fluid Catalytic Cracking; Design, Operation, and Troubleshooting of FCC Facilities” by Reza Sadeghbeigi, published by Gulf Publishing Company, Houston Texas (1995).

Some embodiments of the invention comprise the fluid catalytic cracking of oxygenated hydrocarbon compounds from biological origin. By a hydrocarbon compound is herein preferably understood a compound comprising at least one hydrogen and at least one carbon atom bonded to eachother by at least one covalent bond. By an oxygenated hydrocarbon compound is herein preferably understood a hydrocarbon compound further comprising at least one oxygen atom, which oxygen atom is covalently bonded to at least one carbon atom.

The feed used in some embodiments of the invention comprises oxygenated hydrocarbon compounds from a biological origin. Such compounds from a biological origin may herein also be referred to as bio-feeds or biorenewable feedstocks, as opposed to petroleum-derived feeds and petroleum-derived feedstocks. The hydrocarbon compounds used as a feed in the process of the invention may at least partially be derived from a biological source such as, but not limited to, oil and/or fats from plant sources, including algae and seaweed, fish or animal sources or microbial sources. Preferably the oxygenated hydrocarbon compounds from a biological origin are compounds derived from plant oil, animal fat or used cooking oil. Most preferably the oxygenated hydrocarbon compounds from a biological origin comprise mono-, di- and/or tri-glycerides and/or free fatty acids (FFA's). Such triglycerides and FFA's may for example contain aliphatic hydrocarbon chains in their structure having 9 to 22 carbons.

Plant and animal oils and fats may for example contain 0-30 wt % free fatty acids, which are formed during hydrolysis (e.g. enzymatic hydrolysis) of triglycerides. The amount of free fatty acids present in vegetable oils may for example be 1-5 wt % and in animal fat, 10-25 wt %. The feed used in the process according to the invention may for example include canola oil, corn oil, soy oil, castor oil, cottonseed oil, palm oil, sunflower oil, seaweed oil, tallow oil, fish oil, yellow and brown greases, used cooking oil, and other oils of animal, vegetable or microbial origin.

In a preferred embodiment, the feed in the process according to the invention contains tall oil. Tall oil is a by-product of the wood processing industry. Tall oil may contain rosin esters and rosin acids in addition to FFA's. Rosin acids are cyclic carboxylic acids, rosin esters are the esters thereof. For the process, the feed can include a single oil or a mixture of two or more oils, in any proportions. Triglycerides may be transesterified before use into alkylcarboxylic esters as formiates, acetates etc.

In another preferred embodiment, the feed may contain pyrolysis oil (obtained by pyrolysis (destructive distillation) of biomass in a reactor at temperatures between 400 and 600° C.) or other liquid biocrudes. In another embodiment, the feed may contain a lignocellulosic material, such as for example wood, straw and/or grass.

Preferred biofeeds are liquid biofeeds, especially used cooking oil and tallow oil. The feed in the process of the invention may in addition to the bio-feed comprise a conventional crude oil (also sometimes referred to as a petroleum oil or mineral oil), an unconventional crude oil (that is, oil produced or extracted using techniques other than the traditional oil well method) or a Fisher Tropsch oil (sometimes also referred to as a synthetic oil) and/or a mixture and/or derivates of any of these.

In some embodiemnts of the present invention, in principle, the whole feed may be a biofeed. Suitably the amount of oxygenated hydrocarbon compounds may be up to 65 vol % of the total feed, preferably between 1 and 45 vol %, more preferably between 2 and 35 vol %, even more preferably between 3 and 25 vol % or even between 4 and 15 vol %. The remaining part of the feed may be a petroleum derived feed.

Petroleum derived feeds for the FCC process, which may also be used together with the bio-feeds in the present invention, are preferably high boiling oil fractions, having an initial boiling point of at least 240° C., or even at least 320° C., suitably at least 360° C. or even at least 380° C. (at a pressure of 0.1 MegaPascal). Examples of suitable petroleum derived co-feeds include straight run (atmospheric) gas oils, vacuum gas oil (VGO), flashed distillate, coker gas oils, or atmospheric residue (long residue') and vacuum residue (‘short residue’). Preferred petroleum derived feeds are VGO or long residue. Most preferably heavy gas oils are used, or (high) vacuum gas oils. In addition, high boiling fractions from other refinery units, e.g. the thermal cracker, the hydrocracker and catalytic dewaxing units, may be used.

The feed of some embodiments of the present invention will contain a certain amount of sulphur. That is, the feed in some embodiments of the invention will comprise the oxygenated hydrocarbon compounds from biological origin and an amount of sulphur. The sulphur may be present in any petroleum derived part of the feed and/or in the biofeed. In practice, more than 70 wt % on total sulphur, or even more than 90 wt % on total sulphur, may be originating from a petroleum derived co-feed. The sulphur may be present in the form of organic sulphur, e.g. sulphide, disulphides and/or aromatic sulphur compounds. The sulphur content in the feed may preferably be equal to or less than 6 wt % sulphur based on total feed, more preferably equal to or less than 4 wt %, even more preferably equal to or less than 3 wt %, and most preferably between 0.1 and 2.5 wt %, based on total weight of the feed. Due to the reaction conditions during fluid catalytic cracking, the sulphur present in the feed may for a large part be converted into hydrogen sulphide. Further, mercaptans may be produced.

The hydrogen sulphide will end up in the products stream and may via the main fractionator be separated off as part of the so-called offgas (including dry gas and LPG) or a light fraction as described herein below.

Some embodiments of the invention comprise a step where the feed comprising the oxygenated hydrocarbon compounds from biological origin and an amount of sulphur is contacted with a fluid cracking catalyst at a temperature of equal to or more than 400° C. to produce a products stream. This step may herein also be referred to as FCC or fluid catalytic cracking step. Such an FCC step may suitably be carried out in a so-called FCC unit, suitably in a FCC reactor. This FCC unit may comprise one or more FCC reactor(s) (preferably so-called riser reactor(s)); one or more regenerators; and one or more separators. The separators may include separators for separating the catalyst and a so-called main fractionator to separate the products stream into several fractions.

For example, preheated feed, preheated preferably to a temperature between 160 and 420° C., more preferably between 180 and 380° C., may be injected into a riser reactor, where it may be vaporized and cracked into smaller molecules by contacting and mixing with hot fluid cracking catalyst from a regenerator. Preferably a recycle stream from the main fractionator is simultaneously injected into the reactor. Also (transport) steam may be injected into the riser reactor. The cracking reactions may take place in the reactor within a period of between 0.3 and 12 seconds, preferably between 0.6 and 5 seconds. The riser reactor may be an elongated tubular reactor having for example a diameter between 0.2 and 2.5 m, preferably 0.5 to 1.5 meter and a length between 8 and 32 m, preferably between 12 and 24 m.

In one embodiment, the reaction temperature in the riser reactor is preferably between 400 and 750° C., the pressure is preferably between 0.1 and 0.3 MegaPascal. In a preferred embodiment, the feed is contacted with the fluid cracking catalyst at a temperature in the range of from equal to or more than 460° C. to equal to or less than 610° C., and the contact time between the feed and the fluid catalytic catalyst is preferably less than 10 seconds, more preferably between 0.5 to 8 seconds.

In one embodiment, the catalyst/feed weight ratio is preferably between 4 and 50, more preferably between 5 and 35, even more preferably between 6 and 20. The hydrocarbon vapors and/or transportation steam may fluidize the, preferably powdered, catalyst and the mixture of hydrocarbons and catalyst may flow upwards through the riser reactor to enter a separation unit where a products stream comprising cracked hydrocarbons may be separated from the “spent” fluid cracking catalyst.

Separating fluid cracking catalyst from the products stream may preferably be carried out by one or more horizontal and/or vertical cyclones, often in two or more stages. Preferably at least 96wt % of the spent fluid cracking catalyst is removed from the products stream comprising cracked hydrocarbons, preferably 98wt %, more preferably 99wt %. The spent catalyst particles preferably flow down via a stripping unit in which by means of steam stripping further product hydrocarbons may be removed from the spent catalyst particles. From there the spent catalyst particles can be sent to the regenerator unit. The cracking reactions generally produce an amount of carbonaceous material (often referred to as coke) that usually deposit on the catalyst, which may result in a quick reduction of the catalyst activity. The catalyst can be regenerated by burning off the deposited coke with air blown into the regenerator. The amount of coke can for example be between 2 and 10 wt % based on the feed. Hot flue gas may leave the top of the regenerator through one or more stages of cyclones to remove entrained catalyst from the hot flue gas. The temperature in the regenerator is preferably between 640 and 780° C., the pressure is preferably between 0.15 and 0.35 MegaPascal (MPa). The residence time of the catalyst in the regenerator is preferably between five minutes and 2 hours.

The fluid cracking catalyst can be any catalyst known to the skilled person to be suitable for use in a cracking process. Preferably, the fluid cracking catalyst comprises a zeolite. In addition, the fluid cracking catalyst can contain an amorphous binder compound and/or a filler. Examples of the amorphous binder component include silica, alumina, titania, zirconia and magnesium oxide, or combinations of two or more of them. Examples of fillers include clays (such as kaolin).

The zeolite is preferably a large pore zeolite. By a large pore zeolite is herein preferably understood a zeolite comprising a porous, crystalline aluminosilicate structure having a porous internal cell structure on which the major axis of the pores is in the range of 0.62 nanometer to 0.8 nanometer. The axes of zeolites are depicted in the ‘Atlas of Zeolite Structure Types’, of W. M. Meier, D. H. Olson, and Ch. Baerlocher, Fourth Revised Edition 1996, Elsevier, ISBN 0-444-10015-6. Examples of such large pore zeolites include FAU or faujasite, preferably synthetic faujasite, for example, zeolite Y or X, ultra-stable zeolite Y (USY), Rare Earth zeolite Y (=REY) and Rare Earth USY (REUSY). According to the present invention USY is preferably used as the large pore zeolite.

The fluid cracking catalyst can also comprise a medium pore zeolite. By a medium pore zeolite is herein preferably understood a zeolite comprising a porous, crystalline aluminosilicate structure having a porous internal cell structure on which the major axis of the pores is in the range of 0.45 nanometer to 0.62 nanometer. Examples of such medium pore zeolites are of the MFI structural type, for example, ZSM-5; the MTW type, for example, ZSM-12; the TON structural type, for example, theta one; and the FER structural type, for example, ferrierite. According to the present invention, ZSM-5 is preferably used as the medium pore zeolite.

In the process of the present invention steam may be introduced in the process at a number of positions. Thus, steam may be introduced for instance at the lower end of the riser reactor, half way the riser reactor, in the stripper unit and in the transport pipe of spent catalyst to the regenerator. Steam may for example be added to the feed/fluid cracking catalyst and/or to the stripper unit to improve the separation of the catalyst from the products stream. Further the feed to the FCC process may contain a certain amount of water.

The products stream obtained after the separation of the catalyst, for example at a temperature in the range from 400 to 660° C., preferably between 460 and 610° C., and for example at a pressure in the range from 0.1 to 0.3 MegaPascal (MPa), and optionally the vapors from the stripping unit may flow to the lower section of a fractionator (also referred to herein as main fractionator). This fractionator is preferably a distillation column in which the products stream may be separated into fractions. Suitably at least 60 wt % of the products stream from the fluid catalytic process may be introduced into the main fractionator, more suitably at least 80 wt % and preferably the whole products stream is introduced in the main fractionator. In the main fractionator the products can be separated into FCC end-products. The main products include for example offgas (including C1-C4 hydrocarbons), naphtha, gasoline, light cycle oil, a heavier fraction suitable as fuel oil (sometimes two fractions are separated, light fuel oil and heavy fuel oil) and a slurry oil. Some FCC units produce a light and a heavy naphtha fraction. The slurry oil is preferably returned to the riser reactor. Also a part or all of one or more of the heavier fractions may be returned to the riser reactor.

In this manner a light fraction can be separated from the products stream. By a light fraction is preferably understood a fraction comprising one or more C1-C4 compounds. By a Cx compound is herein understood a compound containing x carbon atoms. The light fraction may comprise or consist of the above mentioned offgas or a fraction thereof. In a preferred embodiment of the invention, the light fraction is a fraction comprising hydrogen, nitrogen, hydrogen sulphide and/or one or more C1-C4 compounds.

In an especially preferred embodiment, the catalyst is suitably separated from the products stream and the separated products stream is fractionated in a distillation column into one fraction comprising one or more C1-C4 compounds and at least one further fraction; whereafter the fraction comprising the one or more C1-C4 compounds is preferably further separated into a fraction comprising one or more C1-C2 compounds (also referred to herein as dry gas fraction) and a fraction comprising one or more C3-C4 compounds (also referred to herein as LPG fraction). In addition to the Cx compounds, one or both fractions may also contain hydrogen and/or nitrogen. The dry gas fraction (the fraction comprising one or more C1-C2 compounds) may for example include hydrogen, carbon monoxide, carbon dioxide, methane, ethane, ethene and/or nitrogen. The LPG fraction (the fraction comprising one or more C3-C4 compounds) may for example include propane, propene, butane and butane.

In such a preferred embodiment, the light fraction from the products stream can be obtained by feeding a separated products stream to a distillation column, fractionating the cracked products stream into an offgas fraction comprising C1-C4 compounds and at least one further fraction, optionally followed by separating fraction comprising the C1-C4 fraction into a fraction comprising mainly C1-C2 compounds (i.e. more than 80 mol % based on hydrocarbons) and a fraction comprising mainly C3-C4 compounds (i.e. more than 80 mol % based on hydrocarbons).

The main fractionator offgas is preferably cooled down, in which step a two phase liquid may be formed. The two phase liquid may comprise an oil phase, containing for example the hydrocarbon compounds, and a water phase, suitably containing condensed water. Due to the presence of hydrogen sulphide, the water phase is sometimes also referred to as sour water. The gas/liquid stream is preferably sent to a combined gas/oil/water separator, although also a separated gas/liquid and liquid/liquid separator can be used.

Preferably the offgas fraction comprising C1-C4 compounds is cooled down to obtain a cooled down gas stream and a liquid oil/water condensate, followed by separation of the oil and the water fraction in an oil/water separation step. The cooled down gas stream can be sent to a gas recovery unit or gas concentration unit, preferably to be separated into a dry gas fraction (including for example hydrogen, methane, ethane, ethene, carbon dioxide, carbon monoxide and nitrogen) and an LPG fraction (propane, propene, butane, butane). Optionally saturated and unsaturated compounds may be separated.

The gas fractions, and often also the naphtha fractions, contain a certain amount of sulphur, mainly in the form hydrogen sulphide. To improve product specification and especially to prevent corrosion problems, the hydrogen sulphide (and, if present, also carbon dioxide)is removed, through an amine treating process as described below. The amine treating process may also remove at least a part of any mercaptans or organic sulphides present in the gas streams.

In the gas recovery unit the gas is preferably compressed (for example by the wet gas compressor) to a pressure between 0.5 and 5 MegaPascal (MPa), preferably 1.0 to 2.5 MPa. This suitably results, suitably after cooling, in the formation of compressed gas and liquids. As indicated, the gas and the liquids, an oily fraction comprising the hydrocarbons and an aqueous fraction (sour water fraction), are preferably separated in a combined gas/oil/water separator, although also a separated gas/liquid and liquid/liquid separator can be used. The compressed gas may for example be sent to the lower section of an absorber, also referred to as the primary absorber. A hydrocarbon liquid, for example a naphtha fraction of the main fractionator, can be introduced in the upper section of the primary absorber. From the upper part of the primary absorber subsequently a dry gas stream may be obtained. From the bottom part of the primary absorber a hydrocarbon liquid comprising C3 and/or C4 compounds may be obtained.

The dry gas stream may optionally be introduced in the lower section of a so-called sponge absorber (also referred to as secondary absorber), in which a lean oil is introduced at the top of the absorber and rich oil (containing C3, C4+ compounds) is obtained at the lower part of the absorber. In this way it is assured that the dry gas only contains C2 and lower molecules. The rich oil may be regenerated and the regenerated light products stream may be introduced as feed in the primary absorber. The hydrocarbon liquid comprising C3 and/or C4 compounds obtained from the bottom part of the primary absorber can be either directly or indirectly (via the gas/oil/water separator system) introduced in the upper part of a stripper column. In the stripper column any C1 or C2 compounds, and optionally some C3 compounds, can be removed from the hydrocarbon liquid. The hydrocarbon liquid from the stripper column may be sent to a debutanizer column, in which a C3-C4 fraction is separated from the FCC naphtha product (also referred to as stabilized FCC naphtha). Suitably a liquid C3-C4 stream can be obtained from the debutanizer column and a light, gaseous top fraction. After cooling, the light fraction may yield a gas fraction comprising light compounds and a two phase oil/water fraction. The cooled gas/liquid stream is sent to a combined gas/oil/water separator, although also a separated gas/liquid and liquid/liquid separator can be used. It is also possible to obtain a gaseous C4-minus top fraction from the debutanizer, which fraction is cooled down followed by separation of the three phases as described above.

As explained above, the sulphur present in the feed may for a large part be converted into hydrogen sulphide and this hydrogen sulphide will end up in the products stream and subsequently in the above light fraction. For a number of reasons (for example environmental reasons, oxidation problems, odor problems) the hydrogen sulphide needs to be removed from this light fraction.

In some embodiments of the invention, the hydrogen sulphide is removed from the light fraction by means of an amine treating process, in which amine treating process activated carbon is used to treat at least part of the amine solution used in the amine treater or to treat at least part of the feed to the amine treater. It is possible to treat only part of the light fraction, for example to treat only the dry gas fraction or only the LPG fraction, by means of an amine treating process to remove the hydrogen sulphide. Preferably, however, both the dry gas fraction and the LPG fraction are treated by means of an amine treating process to remove the hydrogen sulphide. It is also possible to treat only a part of a light fraction (for example only a part of a dry gas fraction or LPG fraction), for example equal to or more than 50 vol % or equal to or more than 80 vol % of the light fraction, but preferably the total light fraction is treated in the amine treater.

In a preferred embodiment, there is provided a process for the fluid catalytic cracking of oxygenated hydrocarbon compounds from biological origin, the process comprising contacting a feed comprising the oxygenated hydrocarbon compounds from biological origin and an amount of sulphur with a fluid cracking catalyst at a temperature of equal to or more than 400° C. to equal to or less than 750° C., preferably during a contact time of less than 10 seconds, to produce a products stream containing hydrogen sulphide; separating fluid cracking catalyst from the products stream; feeding the separated products stream to a fractionator (for example a distillation column) and fractionating the products stream into an fraction comprising hydrogen sulphide and one or more C1-C4 compounds and at least one further fraction; separating the fraction comprising hydrogen sulphide and one or more C1-C4 compounds into a fraction comprising one or more C1-C2 compounds and a fraction comprising one or more C3-C4 compounds; and removing hydrogen sulphide from the fraction comprising C1-C2 compounds and/or from fraction comprising C3-C4 compounds by means of an amine treating process, in which amine treating process activated carbon is used to filter part or whole of the amine solution or part or whole of the feed to the amine treater.

In a preferred embodiment, the light fraction from the products stream is a C1-C2 fraction (a fraction containing or consisting of C1-C2 compounds) or a C3-C4 fraction (a fraction containing or consisting of C3-C4 compounds). In a preferred embodiment a C1-C2 and a C3-C4 fraction is obtained. Preferably hydrogen sulphide is removed from such fraction comprising C1-C2 compounds and/or from such fraction comprising C3-C4 compounds. Preferably the full C1-C2 fraction and the full C3-C4 fraction are subjected to the hydrogen sulphide removal process.

A light fraction comprising C1-C4 compounds preferably comprises at least 75 mol % C1-C4 compounds based on hydrocarbon compounds, preferably at least 90 mol %. A light fraction comprising C1-C2 compounds preferably comprises at least 75 mol % C1-C2 compounds based on hydrocarbon compounds, preferably at least 90 mol %. A light fraction comprising C3-C4 compounds preferably comprises at least 60 mol % C3-C4 compounds based on hydrocarbon compounds, preferably at least 80 mol %.

Without wishing to be bound by any kind of theory, it is believed that the formation of stable foams in the amine gas treating process may be due to the presence of products from catalytically cracking triglycerides and/or catalytically cracking of free fatty acids. It is believed that even ppmv (parts per million by volume) of free fatty acids themselves may contribute to the foaming. Without wishing to be bound by any kind of theory, it is therefore believed that the light fraction may further contain one or more products from catalytically cracking triglycerides and/or catalytically cracking of free fatty acids. For example the light fraction may further contain one or more oxygen containing C1-C4 compounds having a biological origin. Such oxygen containing C1-C4 compounds having a biological origin may suitably have a boiling point equal to or less than 64° C. at a pressure of 0.1 MegaPascal. Examples of such oxygen containing C1-C4 compounds having a biological origin include methanol, ethanol, propanol, butanol, formic acid, acetic acid, propionic acid, butanoic acid, acetone, formaldehyde, acetaldehyde and acrylaldehyde. In a preferred embodiment the light fraction is a fraction comprising one or more compounds from biological origin chosen from the group consisting of methanol, acetone, ethanol, propanol, butanol, formic acid, acetic acid, propionic acid, butanoic acid, formaldehyde, acetaldehyde and acrylaldehyde. More preferably the light fraction is a fraction comprising one or more compounds from biological origin chosen from the group consisting of acetone, acetic acid or propionic acid. Again, without wishing to be bound by any kind of theory, it is believed that due to the bio-feed in the FCC step, the concentration of such oxygen containing C1-C4 compounds in a light fraction may have increased compared to a conventional FCC feed and such increased concentration may lead to a different kind of foaming in an amine treating process.

As indicated above, the light fraction (or preferably offgas fraction) contains a certain amount of sulphur, mainly in the form of hydrogen sulphide. As hydrogen sulphide is an undesired constituent in the light fraction, it is to be removed. Preferably 90 mol % of the hydrogen sulphide is removed from a products stream, more preferably 96 mol %, even more preferably 98 mol %, in a hydrogen sulphide removal process.

In some embodiments of the invention, the hydrogen sulphide is removed by means of an amine treating process, in which the light fraction is suitably washed with an alkylamine solution that absorbs the hydrogen sulphide. The rich amine solution containing the absorbed hydrogen sulphide can be regenerated. The amine treating process may comprise an amine gas treating process and/or an amine liquid-liquid treating process.

Amine gas treating, also known as gas sweetening or acid gas removal, refers to a process in which an aqueous solution of one or more alkylamines is used to remove hydrogen sulphide from a gas stream. In addition also carbon dioxide can be removed. Preferred alkylamines are monoethanolamine (MEA), diethanolamine (DEA), methyldiethanolamine (MDEA), diisopropanolamine (DIPA) and diglycolamine (DGA). Optionally also a physical solvent, e.g. sulfolan, may be present. The main equipment pieces in the amine treater are an absorber and a regenerator. In the absorber a downflowing amine solution can absorb hydrogen sulphide and optionally carbon dioxide from an upflowing sour gas stream to produce a sweetened gas stream (no hydrogen sulphide/carbon dioxide) and an amine solution rich in the absorbed sour gasses (also referred to as rich amine solution or rich amine). The resulting rich amine can then be introduced in the top of a regenerator (a stripper with a reboiler) to produce a stripped overhead gas and regenerated or lean amine solution, which regenerated or lean amine solution is recycled to the absorber. Each absorber in an amine treater preferably has its own regenerator, but is also possible to use a common regenerator for a number of absorbers.

The stripped overhead gas from the regenerator suitably comprises concentrated hydrogen sulphide and optionally carbon dioxide. Hydrogen sulphide rich gas may suitably be sent to a Claus process to recover the sulphur as elemental sulphur.

The amine treating process has for example been described in Oilfield Processing of Petroleum, F. Manning and R. E. Thompson, PennWell Publishing Company, Tulsa, Oklahoma; Acid and Sour Gas Treating Processes, S. A. Newman (ed.), Gulf, 1985; Gas Purification, A. L. Kohl, R. B. Nielsen, Gulf Professional Publishing, 1997; EP 13049 and WO 2008/145680.

In the amine treater the absorber is operated at a temperature preferably between 30 and 60° C. and a pressure preferably from 0.5 to 15 MegaPascal (MPa) in order to absorb as much as possible of the acid gases (for example hydrogen sulphide and optionally carbon dioxide) in the amine liquid. The regenerator is preferably operated at a temperature above the temperature of the absorber, preferably a temperature from 110 to 130° C., and a pressure preferably below the pressure of the absorber, preferably from 0.1 to 0.2 MegaPascal (MPa), at the bottom of the regenerator, in order to remove as much as possible of the acid gases (for example hydrogen sulphide and optionally carbon dioxide) from the amine liquid. In some cases a flash vessel may be used. Rich amine solution may be introduced into such flash vessel at a pressure between the pressure of the absorber and the regenerator. Part of the absorbed gasses may come free here. The flashed amine solution can subsequently be sent to the regenerator.

The amine gas treating process is preferred for a so-called dry gas fraction. The amine liquid-liquid treating process is similar to the amine gas treating process, except that a liquid/liquid contactor may be used instead of a gas absorber. A LPG fraction, mainly comprising C3-C4 compounds, is preferably treated by means of an amine treating process comprising a liquid/liquid contactor instead of a gas absorber, as such LPG fraction may suitably be liquid at the pressure used in the amine treating process. This liquid/liquid contactor may be used to remove the hydrogen sulphide and optionally carbon dioxide from the LPG fraction. Preferably a packed or trayed contactor is used. The obtained sweetened LPG stream can be forwarded to an LPG/amine-water separator to remove trace amounts of amine solution. In some embodiments of the invention, the amine treating process uses activated carbon to treat at least part of the amine solution used in the amine treater or to treat at least part of the feed to the amine treater.

Without wishing to be bound by any kind of theory, it is believed that as a result of the presence of the oxygenated hydrocarbon compounds of biological origin in the feed, highly undesired stable foams may form in the amine treaters, which may result in insufficient removal of hydrogen sulphide. As explained before, these foams appear reversible and when the addition of biofeed was stopped, these problems disappeared.

The activated carbon is used to overcome these problems. The activated carbon (sometimes also referred to as active carbon) preferably has a surface area of equal to or more than 250 m2/g, more preferably equal to or more than 500 m2/g, even more preferably equal to or more than 750 m2/g, most preferably equal to or more than 1000 m2/g. Suitably the activated coal may have a surface areal equal to or less than 1500 m2/g. The activated carbon may be produced from carbonaceous source materials such as nuts, nutshells, e.g. coconut shells, peat, wood, coir, lignite coal and petroleum pitch. The activated carbon may be produced by physical reactivation (carbonization followed by activation/oxidation) or chemical activation (impregnation with acid, base or salt before carbonization). The activated carbon may be in the form of powdered activated carbon (for example having a particle size in the range from 1 to 150 micron), granular activated carbon, extruded activated carbon, bead activated carbon, impregnated carbon or polymer coated carbon. The activated carbon may have macropores (>50 nanometer diameter), mesopores (2-50 nanometer diameter) and/or micropores (<2 nanometer diameter). The activated carbon may be present as a bed or a filter (for example as an activated coal bed or an activated coal filter), or applied as a coating on a carrier. A very suitable activated carbon is a hard, small mesh, steam activated carbon, preferably having a broad range of pore diameters. Another preferred activated carbon is lignite based granular activated carbon. Extensive information about activated carbon can be found in Activated Carbon, H. Marsh & F. R. Reinoso, Elsevier, Oxford, UK (2006) and Activated Carbon Adsorption, R. C. Bansal & M. Goyal, CRC Press, Taylor & Francis, Florida USA, (2005).

The use of activated carbon to reduce foaming by the removal of thermal and chemical amine degradation products as well as organic acids and/or iron sulphides is known. This problem may occur after prolonged operation of an amine unit. It is assumed that this kind of foaming is due to contaminants derived from irreversible degradation of the base amine molecule itself. Further pollutants include solids/particulates, hydrocarbons and process chemicals. As indicated above the co-feed of biofuels resulted in the reversible formation of stable foams, sometimes in combination with emulsions, which is clearly different from the above mentioned permanent foaming problems caused by prolonged operation. The activated carbon according to the present invention prevents the formation of stable foam and foams in combination with emulsions, due to the co-processing of biofuel. In this respect it is observed that it is rather surprising that the (co)processing of biofeed results in the reversible formation of stable foams in the amine treaters, after all treating/separation steps in the product processing.

Without wishing to be bound by any kind of theory it is believed that any oxygen containing C1-C4 compounds having a biological origin having a boiling point equal to or less than 64° C. at a pressure of 0.1 MegaPascal, that may be present in the fraction undergoing the amine treating process, may be removed with the help of the activated carbon. Hence, preferably any methanol, ethanol, propanol, butanol, formic acid, acetic acid, propionic acid, butanoic acid, acetone, formaldehyde, acetaldehyde and/or acrylaldehyde from biological origin present is preferably removed with the help of the activated carbon.

In a preferred embodiment an activated carbon filter is used. The activated carbon filter may be a single activated carbon filter or an activated filter package comprising two or three filters. A suitable package may comprise a main filter, either a precoat type or cartridge type, to remove solid particles. The main filter may be followed by an activated carbon filter that removes chemical contaminants. Optionally, a guard filter may be present to prevent fines from the carbon filter to reach the main amine stream.

The filter or filter system may be used to treat a rich amine solution stream or a lean or regenerated amine solution stream, or, when one or more intermediate flash units are present, a flashed amine stream. Preferably, the regenerated or lean amine solution stream is treated as it has operational advantages (the absence of hydrogen sulphide makes replacement of filter easier; no unexpected gas formation due to pressure changes). In another embodiment two filters are used, one in the lean amine solution and one in the rich amine solution or the flashed amine stream.

It is possible to treat a full amine solution stream with the activated carbon. However, it is preferred to treat a 5 to 50vol % slipstream of the amine solution stream, preferably 10 to 25vol % slipstream. The design of any activated carbon bed is preferably such that there is enough residence time and superficial flow velocity to solve the problems caused by the formation of the stable foams and the emulsions. Preferably an empty bed contact time of at least 5 minutes is used, more preferably an empty bed contact time of 10 minutes to 2 hours, most preferably 15 minutes to 1 hour is used. The superficial flow velocity is preferably at least 0.1 cm/s, more preferably between 0.2 cm/s and 5 cm/s, most preferably between 0.3 and 3 cm/s.

Optionally, one or more additional mechanical filters may be present, upstream and/or downstream of the activated carbon (such filters at least remove specific solid particles). By breaking the emulsions and foams in the amine treating process, the amine wash and regenerator processes will operate more regularly and more efficiently so that product sulfur specification can be met and fuel gas can be processed without excessive sulfur oxide air emissions. It is possible to treat all or a portion of the feed to the amine treater so as to remove the precursors to foaming and emulsions formed in the treater. In this way the integrity of the amine solution is better maintained and the foaming precursors are removed at the source.

As indicated above, it is common practice in an FCC process to feed the separated cracked products stream into a distillation column (main fractionator), fractionating the products stream into an offgas fraction comprising C1-C4 compounds and at least one further fraction, separating fraction comprising the C1-C4 fraction into a fraction comprising C1-C2 compounds (dry gas) and a fraction comprising C3-C4 compounds (LPG), and removing hydrogen sulphide from fraction comprising C1-C2 compounds and/or from fraction comprising C3-C4 compounds by means of the amine treating process. In the case that the amine feed is treated with the activated carbon it is possible to treat the C1-C4 feed, and/or the C1-C2 feed, and/or the C3-C4 feed. Preferably the C1-C4 feed or the C3-C4 feed is treated, more preferably the C1-C4 feed. It is possible to treat the full feed stream or a part of the feed stream, suitably at least 35 vol % is treated, preferably at least 70 vol % is treated, more preferably the full stream is treated. The design of the activated bed design may suitably be such that there is enough residence time and superficial flow velocity to solve the problems caused by the formation of the stable foams and the emulsions. The space velocity of the feed stream is suitably between 0.5 and 12 l/l/h, preferably between 1 and 6 l/l/h. The temperature of the feed stream is suitably between 20 and 75° C., preferably between 30 and 60° C. The pressure is suitably between 0.5 and 15.0 Megapascal (MPa), preferably between 1 and 6 MegaPascal (MPa).

If necessary, some embodiments may involve the use of activated carbon to treat the amine feed stream and the use of activated carbon to treat amine solution (i.e. rich and/or lean amine solution). In addition to the use of the actived carbon, one may remove a slipstream of 5 to 10 vol %/day of the amine solution and/or apply a defoaming agent.

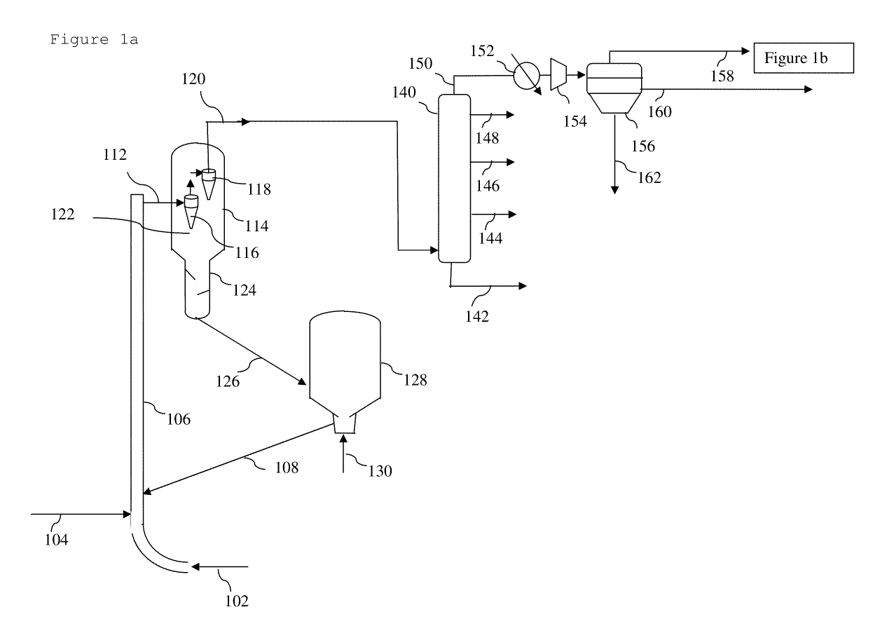

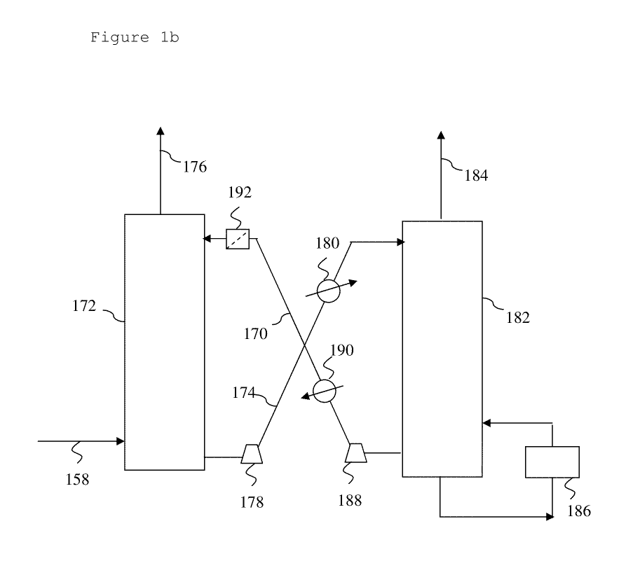

For illustrative puposes, an exemplary embodiment has further been depicted in FIGS. 1a and 1b. In FIG. 1a, a liftgas consisting of steam (102) is introduced into a riser reactor (106). Subsequently a feed containing 90 wt % vacuum gas oil and 10 wt % used cooking oil (104) is introduced. In the bottom of the riser reactor (106), the feed (104) and the steam (102) are mixed with hot regenerated fluid cracking catalyst (108). The mixture of fluid cracking catalyst (108), feed (104) and steam (102) is forwarded into the riser reactor (106). In the riser reactor (106) the feed (104) is catalytically cracked to produce one or more cracked products in a products stream. The mixture (112) comprising one or more cracked products, fluid cracking catalyst and steam is forwarded from the top of the riser reactor (106) into a reactor vessel (114), comprising a first cyclone separator (116) closely coupled with a second cyclone separator (118). A separated products stream (120) is retrieved via the top of the second cyclone separator (118) and forwarded to a fractionator (140). Spent fluid cracking catalyst (122) is retrieved from the bottom of the cyclone separators (116 and 118) and forwarded to a stripper (124) where further cracked products are stripped off the spent fluid cracking catalyst (122). The spent and stripped fluid cracking catalyst (126) is forwarded to a regenerator (128), where the spent fluid cracking catalyst is contacted with air (130) to produce a hot regenerated fluid cracking catalyst (108) that can be recycled to the bottom of the riser reactor (106).

In the fractionator (140) the separated products stream (120) is fractionated into a slurry oil fraction (142), a heavy cycle oil fraction (144), a light cycle oil fraction (146), a naphtha fraction (148) and an offgas fraction (150). The offgas fraction (150) is cooled in cooler (152) and compressed in compressor (154), whereafter it is separated in gas/liquid/liquid separator (156) into a gas phase (158), oil phase (160) and aqueous phase (162).

In FIG. 1b the gas phase (158) comprising H₂S and one or more C1-C4 compounds of biological origin, is contacted with a lean amine solution stream (170) comprising an alkylamine in an absorber (172) at a temperature of about 40° C. In the absorber (172), H₂S is reacted with the alkylamine to produce a stream of H₂S rich amine solution (174) and a stream of treated H₂S lean product gas (176). The stream of H₂S rich amine solution (174) is forwarded via pump (178), heated in heat exchanger (180) and subsequently regenerated in regenerator (182) to produce a stream of H₂S rich product gas (184) and a stream of regenerated H₂S lean amine solution (170). The regenerator is kept at a temperature of about 120° C. by a reboiler (186). The regenerated H₂S lean absorbent composition (170) is cooled in heat exchanger (188) and recycled via pump (190) to absorber (172). Before entering into absorber (172), however, the H₂S lean amine solution (170) is filtered through activated coal filter (192) to remove one or more compounds of biological origin, preferably one or more C1-C4 compounds of biological origin, in order to avoid the formation of a reversible stable foam.

Some embodiments of the invention are further illustrated by the following non-limiting examples.

### EXAMPLES

General

The amine used in the LPG (Liquefied Petroleum Gas) and dry gas washes was methyldiethanolamine (MDEA) in water, at concentrations of 25 wt % and 4.6 wt %, respectively. Accordingly, the nomenclature “LPG amine” and “dry gas amine” means in fact the MDEA solutions in water used to treat the LPG and dry gas, respectively. “LPG amine” and dry gas amine” used in the tests reported here were the same MDEA solutions actually used in the amine treaters in the 3000 bbl/day fully integrated FCC unit.

The concentrations were determined by GC-MS and their water content by Karl Fischer analysis.

Foam Tests

The glassware was first cleaned with distilled water, rinsed with acetone and throughly dried before each experiment. 50 mL of amine sample was added to the glass gas washing cylinder. Nitrogen was bubbled at the given flow rates through the sample via a central glass tube fitted with frit reaching to the bottom of the glass cylinder. This created foaming of the amine. The foam level was allowed to stabilise at a certain height which was then read off for that nitrogen flow rate. The nitrogen flow was stopped, and the time measured for the foam to collapse so that there were no more bubbles in the amine solution. The time required is the “breaking down time” (Bt). These tests were done in duplicate for each amine sample. The average of the 2 measurements is reported.

Filtering Amine

The amine was filtered through a 0.45 μm Whatman FLHP filter to remove any suspended solids. The foam tests were then repeated as described above.

Effect of Activated Carbon Filter

The glassware was first cleaned with distilled water, rinsed with acetone and throughly dried before each experiment. The amine was filtered through activated carbon. Activated carbon GAC 830 from Norit was used.

The activated carbon was rinsed with distilled water to remove any fines. Sufficient activated carbon to fill the burette equipped with a frit was added to a glass beaker and stirred. Any fines float to the top of the water. The water was refreshed and the procedure repeated until the water remained clear. The filter tube was filled with water and the activated carbon poured in ensuring that the activated carbon remained submerged.

The length of the burette was 45 cm above the frit and had an internal diameter of 2 cm. A bed of activated carbon was made with height of ca. 25 cm. The water was drained off to give the bed of activated carbon in the burette. The bulk density of the GAC 830 Norit was measured at 48 g per 100 mL with many voids visible. 100 mL amine was added to the burette and allowed to stand for 2 hours. The amine was slowly (dropwise) drained off from the burette. The first 30 mL of amine was discarded. The rest was collected and filtered through the 0.45 μm Whatman FLHP filter to remove any fines originating from the activated carbon. This amine was then subject to the foam testing as described above. The results for “LPG amine” are presented in Tables 1 and 2 and the results for “dry gas amine” in Table 3.

TABLE 1
“LPG amine” (Test run 6)
(Fh = Foam height; Bt = Breakdown time)

| | 5% | | 25% | | | | | | | | | | |
|---|---|---|---|---|---|---|---|---|---|---|---|---|---|
| | fresh | | fresh | | | | | | | | LPG Amine | | |
| N₂ | MDEA | | MDEA | | | | | LPG Amine | | | active carbon/ | | |
| flow | in water | | in water | | LPG Amine | | | filtered | | | filtrated | | |
| rate | Fh | Bt | Fh | Bt | Fh | Fh | Bt | Fh | Fh | Bt | Fh | Fh | Bt |
| (L/h) | (mL) | (s) | (mL) | (s) | (mL) | (mm) | (s) | (mL) | (mm) | (s) | (mL) | (mm) | (s) |
| 0.0 | 54 | 0 | 52 | 0 | 56 | 50 | 0 | 51 | 45 | 0 | 52 | 46 | 0 |
| 22.8 | 66 | 9 | 58 | 2 | 66 | 63 | 3 | 60 | 85 | 2 | 56 | 52 | 1 |
| 37.5 | 126 | 23 | 118 | 40 | 86 | 76 | 100 | 96 | 95 | 71 | 84 | 67 | 10 |
| 68.3 | 230 | 25 | 146 | 40 | 88 | 73 | 95 | 94 | 80 | 64 | 88 | 65 | 10 |
| 91.0 | 255 | 29 | 158 | 41 | 86 | 68 | 80 | 92 | 70 | 60 | 96 | 64 | 14 |

The fresh 5 wt % MDEA in water has a higher foam height than 25 wt % solution. it can be clearly seen from the Table 1 above that the breakdown time of the LPG amine is progressively reduced in the order no treatment >filtered >treated by filtration through activated carbon +filtering.

TABLE 2
“LPG amine” (Test run 5)

| | | | | | LPG | |
|---|---|---|---|---|---|---|
| | | | LPG | | Amine active | |
| N₂ | LPG Amine | | Amine filtered | | carbon/filtered | |
| flow | Foam | | Foam | | Foam | Break- |
| rate | height | Breakdown | height | Breakdown | height | down |
| (L/h) | (mL) | time (s) | (mL) | time (s) | (mL) | time (s) |
| 0.0 | 52 | 0 | 52 | 0 | 53 | 0 |
| 22.8 | 60 | 2 | 70 | 18 | 58 | 3 |
| 37.5 | 190 | 60 | 160 | 41 | 100 | 16 |
| 68.3 | 190 | 55 | 132 | 50 | 110 | 18 |
| 91.0 | 200 | 70 | 120 | 52 | 108 | 19 |

It can be seen from the Table 2 above that the breakdown time of the LPG amine is progressively reduced in the order no treatment>filtered>treated by filtration through activated carbon+filtering.

The foam height is also significantly reduced.

TABLE 3
“Dry gas amine” (Test Run 6)

| | | | | | Dry Gas amine | |
|---|---|---|---|---|---|---|
| | | | Dry Gas Amine | | active | |
| N₂ | Dry gas Amine | | filtered | | carbon/filtered | |
| flow | Foam | | Foam | | Foam | Break- |
| rate | height | Breakdown | height | Breakdown | height | down |
| (L/h) | (mL) | time (s) | (mL) | time (s) | (mL) | time (s) |
| 0.0 | 52 | 0 | 53 | 0 | 52 | 0 |
| 22.8 | 90 | 34 | 110 | 50 | 70 | 10 |
| 37.5 | 170 | 55 | 260 | 60 | 160 | 25 |
| 68.3 | 190 | 65 | 300 | 60 | 230 | 26 |
| 91.0 | 200 | 75 | 320 | 70 | 245 | 33 |

It can be seen from the Table 3 above that the treatment of the dry gas amine with activated carbon substantially reduces the foam height and breakdown time.

Therefore, embodiments of the present invention are well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, substituted, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount whether accompanied by the term “about” or not. In particular, the phrase “from about a to about b” is equivalent to the phrase “from approximately a to b,” or a similar form thereof. Also, the terms in the claims have their plain, ordinary meaning unless otherwise explicitly and clearly defined by the patentee. Moreover, the indefinite articles “a” or “an,” as used in the claims, are defined herein to mean one or more than one of the element that it introduces. If there is any conflict in the usages of a word or term in this specification and one or more patent or other documents that may be incorporated herein by reference, the definitions that are consistent with this specification should be adopted.

1. A process for the fluid catalytic cracking of oxygenated hydrocarbon compounds from biological origin, the process comprising
contacting a feed comprising the oxygenated hydrocarbon compounds from biological origin and an amount of sulphur with a fluid cracking catalyst at a temperature of equal to or more than 400° C. to produce a products stream;
separating the fluid cracking catalyst from the products stream and separating a light fraction from the products stream; and
removing hydrogen sulphide from the light fraction by means of an amine treating process, wherein activated carbon is used to treat at least part of an amine solution used in the amine treating process or to treat at least part of the feed to the amine treating process.

2. The process of claim 1, wherein the oxygenated hydrocarbon compounds from biological origin are derived from plant oil, animal fat or used cooking oil.

3. The process of claim 1, wherein the amount of oxygenated hydrocarbon compounds is up to 65 vol % of the total feed.

4. The process of claim 1 wherein the amount of oxygenated hydrocarbon compounds is between 1 and 45 vol %.

5. The process of claim 1 wherein the amount of oxygenated hydrocarbon compounds is between 2 and 35 vol %.

6. The process of claim 1 wherein the amount of oxygenated hydrocarbon compounds is between 3 and 25 vol %.

7. The process of claim 1, wherein the light fraction is a fraction of the products stream comprising oxygen containing C1-C4 compounds having a biological origin having a boiling point equal to or less than 64° C.

8. The process of claim 1, wherein the light fraction is a fraction comprising methanol, ethanol, propanol, butanol, formic acid, acetic acid, propionic acid, butanoic acid, acetone, formaldehyde, acetaldehyde and/or acrylaldehyde.

9. The process of claim 1, wherein the light fraction is a C1-C2 fraction and/or a C3-C4 fraction.

10. The process of claim 1, wherein the light fraction from the cracked products stream is obtained by feeding separated cracked products stream to a distillation column, fractionating the cracked products stream into an offgas fraction comprising C1-C4 compounds and at least one further fraction, optionally followed by separating fraction comprising the C1-C4 fraction into a fraction comprising mainly C1-C2 compounds and a fraction comprising mainly C3-C4 compounds.

11. The process of claim 9, wherein hydrogen sulphide is removed from a fraction comprising C1-C2 compounds and/or from a fraction comprising C3-C4 compounds.

12. The process of claim 1, wherein steam is added to the feed/fluid cracking catalyst or steam is used to improve the separation of the catalyst from the products stream.

13. The process of claim 1, wherein the surface area of the activated carbon in the filter is more than 500 m²/g.

14. The process of claim 1, wherein the activated carbon in the filter is physically or chemically activated.

15. The process of claim 1, wherein a slipstream of the amine solution is treated.

16. The process of claim 1 wherein a 5 to 50% slipstream of the amine solution is treated.

17. The process of claim 1 wherein a 10 to 25% slipstream of the amine solution is treated.

18. The process of claim 15, wherein the amine solution is a lean amine solution from the regenerator.

19. The process of claim 1, wherein one or more antifoaming agents are used in the amine treating process.

20. The process of claim 1, wherein a slipstream of 5 to 10 vol %/day of the amine solution is removed from the process and replaced with fresh amine solution.
